(19) United States
(12) Patent Application Publication
(10) Pub. No.: US 2024/0225495 A1
(43) Pub. Date: Jul. 11, 2024

(54) SYSTEM AND METHOD FOR NON-INVASIVE MEASUREMENT OF GLYCATED HEMOGLOBIN

(71) Applicant: KOREA I.T.S. CO., LTD., Seoul (KR)

(72) Inventors: Ki Doo KIM, Seoul (KR); Hossain SHIFAT, Seoul (KR)

(73) Assignee: KOREA I.T.S. CO., LTD., Seoul (KR)

(21) Appl. No.: 18/577,473

(22) PCT Filed: Jun. 19, 2020
(86) PCT No.: PCT/KR2020/007958

§ 371 Date: Jan. 8, 2024

(30) Foreign Application Priority Data
Apr. 13, 2020 (KR) 10-2020-0044687
May 11, 2020 (KR) 10-2020-0056039

(51) Int. Cl. A61B 5/1455; A61B 5/00

(52) U.S. Cl. CPC A61B 5/14551; A61B 5/0059

(57) ABSTRACT

The present disclosure relates to a system and method for the non-invasive measurement of glycated hemoglobin, wherein the method includes: irradiating a measurement subject with first to third lights having different wavelength values by means of first to third LED modules positioned on one side of the body of the measurement subject; using a light detection unit positioned corresponding to the first to third LED modules to detect first to third derived lights derived from the first to third lights by passing through the measurement subject; generating first and second ratio equations, respectively, for first and second derived light sets composed of two of the first to third derived lights; and calculating concentrations of glycated hemoglobin (HbA1c) and arterial blood oxygen saturation (SpO2) of the measurement subject by combining the first and second ratio equations.

### TECHNICAL FIELD

The present disclosure relates to a system and method for non-invasive measurement of glycated hemoglobin, and more specifically, to a system and method for non-invasive measurement of glycated hemoglobin capable of accurately and easily non-invasively measuring the concentration of glycated hemoglobin (HbA1c) using two ratio equations regarding the ratio of attributes according to two wavelengths among multiple different wavelengths that penetrate the blood.

### BACKGROUND ART

Diabetes is a metabolic disease characterized by hyperglycemia caused by dysfunction or secretion of insulin, which is necessary for controlling blood sugar levels in the body. Chronic hyperglycemia due to diabetes causes damage and functional insufficiency in each organ of the body. In particular, the chronic hyperglycemia causes microvascular complications of the retina, kidneys, and nerves, and macrovascular complications such as arteriosclerosis, cardiovascular, and cerebrovascular diseases, resulting in an increase in mortality.

However, diabetes may reduce the worsening or complication rate of diabetes due to blood sugar control, weight loss, and medication. Accordingly, diabetic patients need to frequently measure their own blood sugar levels to manage their blood sugar levels and undergo regular glycated hemoglobin (HbA1C) tests, which are as important a treatment indicator as the blood sugar levels of the diabetic patients.

The glycated hemoglobin (HbA1c) test is a test that determines the extent to which the hemoglobin in red blood cells, which plays a role in transporting oxygen in the blood, has been glycated. Depending on the average lifespan of red blood cells, the test reflects changes in blood sugar over the past 2 to 3 months. Since glucose always exists in normal people, hemoglobin is glycated to some extent in the blood. The normal value varies depending on a test method, but usually up to 5.6% is normal.

In diabetic patients, as the concentration of glucose in the blood increases, glycated hemoglobin, in other words, the level of glycated hemoglobin, also increases. Accordingly, the direction of future treatment is decided by reviewing these results, which clearly reveal the extent of blood sugar control so far.

The conventional method of measuring glycated hemoglobin (HbA1c) is to acquire a capillary blood sample by collecting blood from a vein in the arm of a measurement subject or pricking the tip of the finger with a small and pointed needle, and use the acquired blood to measure the concentration of glycated hemoglobin (HbA1c). The invasive method of measuring glycated hemoglobin has an issue of increasing the burden of blood collection on measurement subjects and providing inaccurate levels in cases of short red blood cell lifespan, pregnancy, or kidney disease.

The background technology of the present disclosure is disclosed in Korean Patent Application Publication No. 10-2019-0037254 (published on Apr. 5, 2019).

### RELATED ART DOCUMENT

### Patent Document

Korean Patent Application Publication No. 10-2019-0037254 (Apr. 5, 2019)

### DETAILED DESCRIPTION OF THE INVENTION

### Technical Problem

A technical task of the present disclosure is directed to providing a system and method for non-invasive measurement of glycated hemoglobin capable of accurately and easily non-invasively measuring the concentration of glycated hemoglobin (HbA1c) using two ratio equations regarding the rate of attributes according to two wavelengths among multiple different wavelengths that penetrate the blood using Photon-Diffusion Theory or Beer-Lambert Law.

### Technical Solution

A method for non-invasive measurement of glycated hemoglobin according to an embodiment of the present disclosure includes: irradiating a measurement subject with first to third lights having different wavelength values by means of first to third LED modules positioned on one side of the body of the measurement subject; using a light detection unit positioned corresponding to the first to third LED modules to detect first to third derived lights derived from the first to third lights by passing through the measurement subject; generating first and second ratio equations, respectively, for first and second derived light sets composed of two of the first to third derived lights; and calculating concentrations of glycated hemoglobin (HbA1c) and arterial blood oxygen saturation (SpO2) of the measurement subject by combining the first and second ratio equations.

The light detection unit may be positioned at an opposite side or on the same side surface relative to positions of the first to third LED modules.

One side of the body of the measurement subject may include a site where capillaries existing under the skin are able to be sensed, depending on the thickness of the skin.

The generation may include generating the first and second ratio equations using Photon-Diffusion Theory or Beer-Lambert Law.

The generation may include generating an attribute equation for each wavelength of the first to third derived lights according to the Photon-Diffusion Theory, and generating the first and second ratio equations using the attribute equation for each derived light of the first and second derived light sets.

The generation may include generating an equation for transmittance or reflectance including a total absorption coefficient and a scattering coefficient for each wavelength of the first to third derived lights as the attribute equation.

The generation may include: applying the total absorption coefficient and scattering coefficient to spherical geometry to express the transmittance or reflectance of each of the first to third derived lights using a mathematical equation; generating a ratio for the transmittance or reflectance for each derived light of the first derived light set using the first ratio equation; and generating a ratio for the transmittance or reflectance for each derived light of the second derived light set using the second ratio equation.

The calculation may include: generating first and second conversion formulas by applying the glycated hemoglobin (HbA1c) and the arterial blood oxygen saturation (SpO2) as unknown to each of the first and second ratio equations; applying coefficient values acquired corresponding to first to third wavelength ranges to each of the first and second conversion formulas; and combining the first and second conversion formulas to convert the concentrations of the glycated hemoglobin (HbA1c) and the arterial blood oxygen saturation (SpO2) into each functional formula regarding the first and second ratio equations.

The generation may include applying attribute ratios regarding each derived light of each of the first and second derived light sets and the first to third derived lights measured by the light detection unit to the Beer-Lambert Law to generate the first and second ratio equations. The generation may include: generating the first ratio equation representing a ratio of absorbance for each derived light of the first derived light set by applying the Beer-Lambert Law; and generating the second ratio equation representing a ratio of absorbance for each derived light of the second derived light set by applying the Beer-Lambert Law.

The calculation may include applying the first to third derived lights measured by the light detection unit to the first and second ratio equations to compute the concentrations of the glycated hemoglobin (HbA1c) and the arterial blood oxygen saturation (SpO2) of the measurement subject.

The calculation may include: generating first and second conversion formulas by applying the glycated hemoglobin (HbA1c) and the arterial blood oxygen saturation (SpO2) as unknown to each of the first and second ratio equations; applying a molar extinction coefficient when applying first to third wavelengths to each of the first and second conversion formulas; and combining the first and second conversion formulas to convert the concentrations of the glycated hemoglobin (HbA1c) and the arterial blood oxygen saturation (SpO2) into each functional formula regarding the first and second ratio equations.

A system for non-invasive measurement of glycated hemoglobin according to another embodiment of the present disclosure includes: first to third LED modules positioned on one side of the body of a measurement subject and respectively irradiating the measurement subject with first to third lights having different wavelength values; a light detection unit positioned corresponding to the first to third LED modules to detect first to third derived lights derived from the first to third lights by passing through the measurement subject; and a computation unit that generates first and second ratio equations, respectively, for first and second derived light sets composed of two of the first to third derived lights and calculates concentrations of glycated hemoglobin (HbA1c) and arterial blood oxygen saturation (SpO2) of the measurement subject by combining the first and second ratio equations.

The light detection unit may be positioned at an opposite side or on the same side surface relative to the positions of the first to third LED modules.

One side of the body of the measurement subject may include a site where capillaries existing under the skin are able to be sensed, depending on the thickness of the skin.

The computation unit may generate the first and second ratio equations using Photon-Diffusion Theory or Beer-Lambert Law.

When using the Photon-Diffusion Theory, the computation unit may calculate the concentration of the glycated hemoglobin using transmittance when the light detection unit is positioned at the opposite side, and calculate the concentration of the glycated hemoglobin using reflectance when the light detection unit is positioned on the same side surface.

The computation unit may calculate the concentration of the glycated hemoglobin using the Beer-Lambert Law and using absorbance when the light detection unit is positioned at the opposite side.

### Effects of the Invention

The disclosed technology may have the following effects. However, it does not mean that a specific exemplary embodiment should include the entire following effects or should include only the following effects, and it should not be understood that the scope of right of disclosed technology is limited thereto.

According to an embodiment of the present disclosure as described above, the concentration of glycated hemoglobin (HbA1C) can be measured non-invasively using the intensity change rate of LED light having three different wavelengths, and the concentrations of glycated hemoglobin (HbA1C) and arterial blood oxygen saturation (SpO2) can be measured accurately and easily through the intensity change rate of light to which the transmittance of LED light is applied or the intensity change rate of light to which reflectance of LED light is applied according to the Photon-Diffusion Theory.

According to an embodiment of the present disclosure as described above, the concentration of glycated hemoglobin (HbA1C) can be measured non-invasively using the intensity change rate of LED light having three different wavelengths, and the concentrations of glycated hemoglobin (HbA1C) and arterial blood oxygen saturation (SpO2) can be measured accurately and easily through the absorbance of LED light calculated by the Beer-Lambert Law.

### BRIEF DESCRIPTION OF THE DRAWINGS

FIG. 1 is a configuration diagram illustrating a system for non-invasive measurement of glycated hemoglobin according to an embodiment of the present disclosure.

FIG. 2 is an example diagram for explaining transmitted light and reflected light generated when a body formed in a hemispherical shape, such as a finger, is irradiated with an LED.

FIG. 3 is a flowchart for explaining a method for measuring glycated hemoglobin according to an embodiment of the present disclosure.

FIGS. 4 and 5 are example diagrams illustrating the system for non-invasive measurement of glycated hemoglobin according to an embodiment of the present disclosure installed on a finger.

FIGS. 6 and 7 are flowcharts for explaining stage S330 illustrated in FIG. 3.

FIG. 8A is a graph showing PPG signals obtained by measuring photoblood flow in a portion of the body of a measurement subject.

FIG. 8B is a diagram for explaining a transmission distance (d) of LED light when blood enters capillaries.

FIG. 8C is a diagram for explaining a transmission distance (d) of LED light when blood passes through the capillaries.

### BEST MODE FOR CARRYING OUT THE INVENTION

The explanation of the present disclosure is merely an embodiment for structural or functional explanation, so the scope of the present disclosure should not be construed to be limited to the embodiments explained in the embodiment. That is, since the embodiments may be implemented in several forms without departing from the characteristics thereof, it should also be understood that the described embodiments are not limited by any of the details of the foregoing description, unless otherwise specified, but rather should be construed broadly within its scope as defined in the appended claims. Therefore, various changes and modifications that fall within the scope of the claims, or equivalents of such scope are therefore intended to be embraced by the appended claims.

Terms described in the present disclosure may be understood as follows.

While terms such as “first”, “second”, etc., may be used to describe various components, such components must not be understood as being limited to the above terms. The above terms are used to distinguish one component from another. For example, a first component may be referred to as a second component without departing from the scope of rights of the present disclosure, and likewise a second component may be referred to as a first component.

It will be understood that when an element is referred to as being “connected to” another element, it may be directly connected to the other element or intervening elements may also be present. In contrast, when an element is referred to as being “directly connected to” another element, no intervening elements are present. In addition, unless explicitly described to the contrary, the word “comprise” and variations such as “comprises” or “comprising” will be understood to imply the inclusion of stated elements but not the exclusion of any other elements. Meanwhile, other expressions describing relationships between components such as “between”, “immediately between” or “adjacent to” and “directly adjacent to” may be construed similarly.

Singular forms “a”, “an” and “the” in the present disclosure are intended to include the plural forms as well, unless the context clearly indicates otherwise. It will be further understood that terms such as “including” or “having”, etc., are intended to indicate the existence of the features, numbers, operations, actions, components, parts, or combinations thereof disclosed in the specification, and are not intended to preclude the possibility that one or more other features, numbers, operations, actions, components, parts, or combinations thereof may exist or may be added.

In each phase, reference numerals (for example, a, b, c, etc.) are used for the sake of convenience in description, and such reference numerals do not describe the order of each phase. The order of each phase may vary from the specified order, unless the context clearly indicates a specific order. In other words, each phase may take place in the same order as the specified order, may be performed substantially simultaneously, or may be performed in a reverse order.

The present disclosure may be implemented as machine-readable codes on a machine-readable medium. The machine-readable medium may include any type of recording device for storing machine-readable data. Examples of the machine-readable recording medium may include a read-only memory (ROM), a random access memory (RAM), a compact disk-read only memory (CD-ROM), a magnetic tape, a floppy disk, optical data storage, or any other appropriate type of machine-readable recording medium. The medium may also be carrier waves (for example, Internet transmission). The computer-readable recording medium may be distributed among networked machine systems which store and execute machine-readable codes in a de-centralized manner.

The terms used in the present application are merely used to describe particular embodiments, and are not intended to limit the present disclosure. Unless otherwise defined, all terms used herein, including technical or scientific terms, have the same meanings as those generally understood by those with ordinary knowledge in the field of art to which the present disclosure belongs. Such terms as those defined in a generally used dictionary are to be interpreted to have the meanings equal to the contextual meanings in the relevant field of art, and are not to be interpreted to have ideal or excessively formal meanings unless clearly defined in the present application.

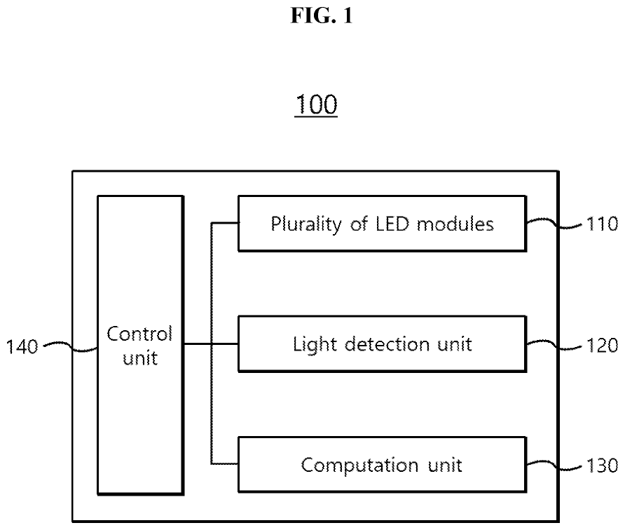

FIG. 1 is a configuration diagram illustrating a system for non-invasive measurement of glycated hemoglobin according to an embodiment of the present disclosure.

Referring to FIG. 1, a system 100 for non-invasive measurement of glycated hemoglobin may include a plurality of LED modules 110, a light detection unit 120, a computation unit 130, and a control unit 140.

First, the plurality of LED modules 110 and the light detection unit 120 may be positioned on one side of a portion of the body of a subject. In addition, the light detection unit 120 may detect transmitted or reflected light irradiated from the plurality of LED modules 110.

The plurality of LED modules 110 may be composed of at least three LEDs, and according to an embodiment of the present disclosure, the plurality of LED modules 110 may include a first LED module 111 having a first wavelength value, a second LED module 112 having a second wavelength value, and a third LED 113 having a third wavelength value. Herein, the first to third wavelength values may correspond to different values. In other words, the plurality of LED modules 110 may respectively irradiate a measurement subject with first to third lights having different wavelength values in a state of being positioned on one side of the body of the measurement subject.

The light detection unit 120 may measure the intensity of light transmitted through blood in the human body or reflected by the human body from the first to third LED modules 111 to 113. The light detection unit 120 may be installed at a point opposite the plurality of LED modules 110 to measure the intensity of transmitted light, or may be positioned on the same side as the plurality of LED modules 110 to measure the intensity of reflected light.

Herein, the light detection unit 120 may be positioned at an opposite side or on the same side surface relative to positions of the plurality of LED modules 110, and does not detect transmitted light and reflected light simultaneously, but may selectively detect the same according to the positional relationship between the body portion where the light detection unit 120 is installed and the plurality of LED modules 110. In other words, the light detection unit 120 may perform an operation of detecting first to third derived lights derived from the first to third lights by passing through the measurement subject in a state of being positioned corresponding to the first to third LED modules 110.

In an embodiment, one side of the body of the measurement subject where the plurality of LED modules 110 and the light detection unit 120 are positioned may include a site where capillaries existing under the skin may be sensed depending on the thickness of the skin. For example, one side of the body of the measurement subject may include fingers, wrist, forehead, cheek, ear, etc., but is not necessarily limited thereto, and may include various body portions depending on installation conditions.

The computation unit 130 generates first and second ratio equations for each of the first and second derived light sets composed of two of the first to third derived lights, and calculates the concentrations of glycated hemoglobin (HbA1c) and arterial blood oxygen saturation (SpO2) of the measurement subject by combining the first and second ratio equations.

The control unit 140 may control the overall operation of the system 100 for non-invasive measurement of glycated hemoglobin and manage the control flow or data flow among the plurality of LED modules 110, the light detection unit 120, and the computation unit 130.

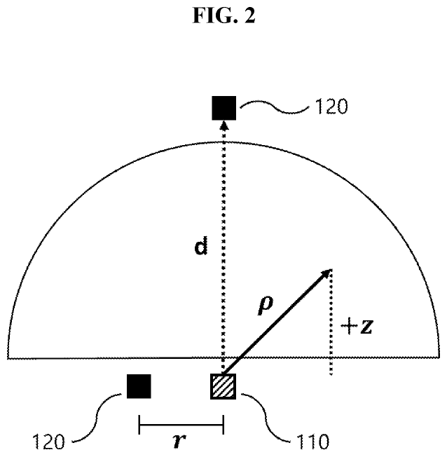

FIG. 2 is an example diagram for explaining transmitted light and reflected light generated when a body formed in a hemispherical shape, such as a finger, is irradiated with an LED.

Referring to FIG. 2, the light detection unit 120 may be installed at a point opposite to the plurality of LED modules 110 when measuring glycated hemoglobin using the transmittance (or absorbance) of LED light, and the light detection unit 120 may be installed to be positioned on the same side surface as the plurality of LED modules 110 when measuring glycated hemoglobin using the reflectance of LED light.

In an embodiment, the computation unit 130 may more accurately measure the concentrations of glycated hemoglobin (HbA1C) and arterial blood oxygen saturation (SpO2) in consideration of the transmittance and reflectance that occur when the dermis is irradiated with light from the plurality of LED modules 110 according to the Photon-Diffusion Theory. More specifically, the computation unit 130 may calculate the concentration of glycated hemoglobin using the transmittance when the light detection unit 120 is positioned at an opposite side, and calculate the concentration of glycated hemoglobin using the reflectance when the light detection unit 120 is positioned on the same side surface.

In another embodiment, the computation unit 130 may accurately measure the concentrations of glycated hemoglobin (HbA1C) and arterial blood oxygen saturation (SpO2) in consideration of the absorbance that occur when the dermis is irradiated with light from the plurality of LED modules 110 according to the Beer-Lambert Law. More specifically, the computation unit 130 may calculate the concentration of glycated hemoglobin using the absorbance when the light detection unit 120 is positioned at an opposite side.

The plurality of LED modules 110 and the light detection unit 120 according to an embodiment of the present disclosure may be installed in a body portion where capillaries existing under the skin may be sensed depending on the thickness of the skin. For example, the plurality of LED modules 110 and the light detection unit 120 may be installed on one of the fingers, wrist, forehead, cheek, or ear of the measurement subject, and it is assumed that the surfaces of the fingers and wrist are formed in a spherical shape. Accordingly, the system 100 for non-invasive measurement of glycated hemoglobin according to an embodiment of the present disclosure may acquire an mathematical equation for transmittance or reflectance using spherical geometry.

As illustrated in FIG. 2, the computation unit 130 may acquire flux density for the distance value (d, r) from the plurality of LED modules 110 to the light detection unit 120 according to spherical geometry based on the fact that the body portion to be measured is formed as a sphere rather than a plane.

Next, the computation unit 130 may calculate the transmittance or reflectance using the acquired flux density of the LED light.

In other words, the computation unit 130 may acquire ratio equations for transmittance or reflectance according to two wavelengths among a plurality of different wavelengths. In other words, the computation unit 130 may generate first and second ratio equations for each of the first and second derived light sets composed of two of the first to third derived lights.

In addition, the computation unit 130 may acquire a first ratio equation R1 representing the ratio of the transmittance (or absorbance) corresponding to the second LED module 112 and the transmittance (or absorbance) corresponding to the third LED module 113, and a second ratio equation R2 representing the ratio of the transmittance (or absorbance) corresponding to the first LED module and the transmittance (or absorbance) corresponding to the third LED module 113.

In addition, the computation unit 130 may acquire a first ratio equation representing the ratio of the reflectance corresponding to the second LED module 112 and the reflectance corresponding to the third LED module 113, and a second ratio equation representing the ratio of the reflectance corresponding to the first LED module and the reflectance corresponding to the third LED module 113.

Next, the computation unit 130 may apply the intensity of light radiated from the first to third LED modules 110 and the intensity of light measured by the light detection unit 120 to the first ratio equation and the second ratio equation, respectively, and may compute the concentration of glycated hemoglobin (HbA1c) and arterial blood oxygen saturation (SpO2) of the measurement subject.

Light installed and radiated on a portion of the body of a subject may not only penetrate the blood, but may also penetrate the skin tissue of the body. In this connection, the dermis is a homogeneous mixture containing arterial blood, venous blood, water, and other contents, which causes light absorption in the blood and water and scattering in the other contents.

Accordingly, the system 100 for non-invasive measurement of glycated hemoglobin according to an embodiment of the present disclosure may measure glycated hemoglobin using the transmittance or reflectance of LED light according to Photon-Diffusion Theory. In addition, the system 100 for non-invasive measurement of glycated hemoglobin according to another embodiment of the present disclosure may measure glycated hemoglobin using the absorbance of LED light according to Beer-Lambert Law.

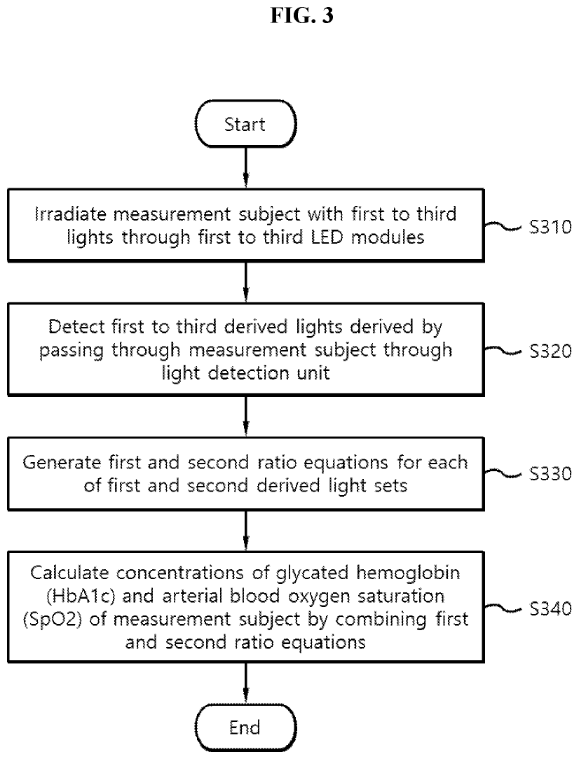

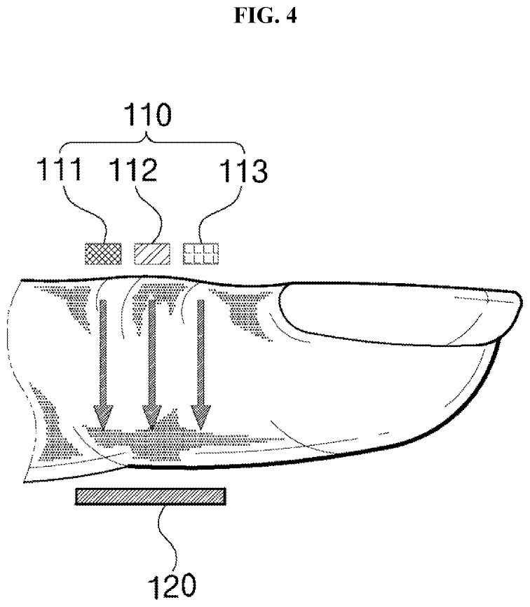

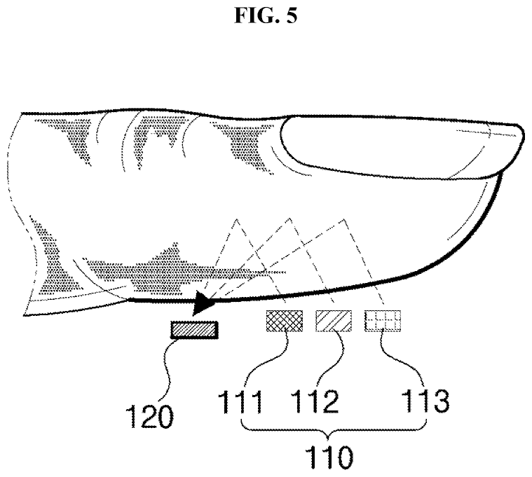

FIG. 3 is a flowchart for explaining a method for measuring glycated hemoglobin according to an embodiment of the present disclosure. FIGS. 4 and 5 are example diagrams illustrating the system for non-invasive measurement of glycated hemoglobin according to an embodiment of the present disclosure installed on a finger.

Referring to FIGS. 3 to 5, in order to non-invasively measure blood sugar, the plurality of LED modules 110 and the light detection unit 120 may be installed on one side of the body of the measurement subject opposite to each other or on the same side surface. Herein, one side of the body of the measurement subject may correspond to a site where capillaries existing under the skin are able to be sensed, depending on the thickness of the skin. For example, one side of the body of the measurement subject may include fingers, wrist, forehead, cheek, ear, etc.

The plurality of LED modules 110 may include the first LED module 111 that emits green light, the second LED module 112 that emits red light, and the third LED module 113 that emits infrared (IR) light, wherein the wavelength values of the first LED module 111 to the third LED module 113 may be different from each other. In other words, the first LED module 111 may have a first wavelength value (λ₁) the second LED module 112 may have a second wavelength value (λ₂), and the third LED module 113 may have a third wavelength value (λ₃).

In a state where the first LED module 111, the second LED module 112, and the third LED module 113 are positioned on one side of the body of the measurement subject, and the light detection unit 120 is positioned at an opposite side or on the same side surface, by the applied power, the first LED module 111, the second LED module 112, and the third LED module 113 may radiate emitted light, in other words, first to third lights, respectively, toward the measurement subject (stage S310). As a result, the first to third lights may be radiated in the direction of the light detection unit 120.

Then, the light detection unit 120 may detect the first to third derived lights derived by passing through the measurement subject (stage S320). In other words, the light detection unit 120 may measure the intensity of light radiated from the first LED module 111, the second LED module 112, and the third LED module 113 and transmitted or reflected. In this connection, since the wavelength values radiated from the first LED module 111, the second LED module 112, and the third LED module 113 are different, the intensity of light measured by the light detection unit 120 may be measured differently for each LED module.

Next, the computation unit 130 may acquire the intensity value of light measured from the light detection unit 120. The computation unit 130 may acquire the intensity value of the light radiated from each of the first to third LED modules 110.

In other words, the first to third lights radiated toward the measurement subject (for example, a finger) in stage S310 may pass through a specific point of the finger or be reflected at a specific point and proceed. As a result, the intensity of the derived light after the LED light of the first LED module 111, the second LED module 112, and the third LED module 113 is transmitted (or absorbed) or reflected by the finger may be detected by the light detection unit 120. The computation unit 130 may acquire the intensity of light for each of the first LED module 111, the second LED module 112, and the third LED module 113.

Next, the computation unit 130 may acquire two ratio equations (stage S330).

In addition, the computation unit 130 may acquire an equation for the ratio of each transmittance (or absorbance) when the second and third lights are transmitted through the second LED module 112 and the third LED module 113 and the ratio of each transmittance (or absorbance) when the first and third lights are transmitted through the first LED module 111 and the third LED module 113.

In addition, when the second and third lights are reflected through the second LED module 112 and the third LED module 113, the computation unit 130 may acquire an equation for the ratio of each reflectance and the ratio of each reflectance when the first and third lights are reflected through the first LED module 111 and the third LED module 113.

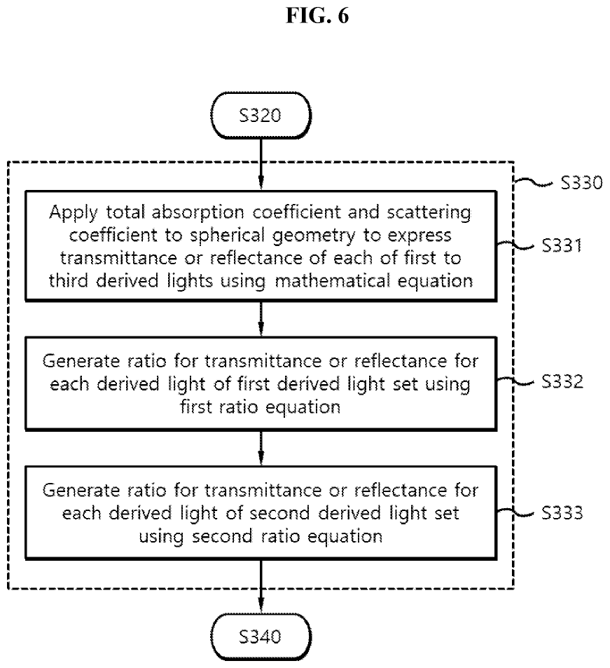

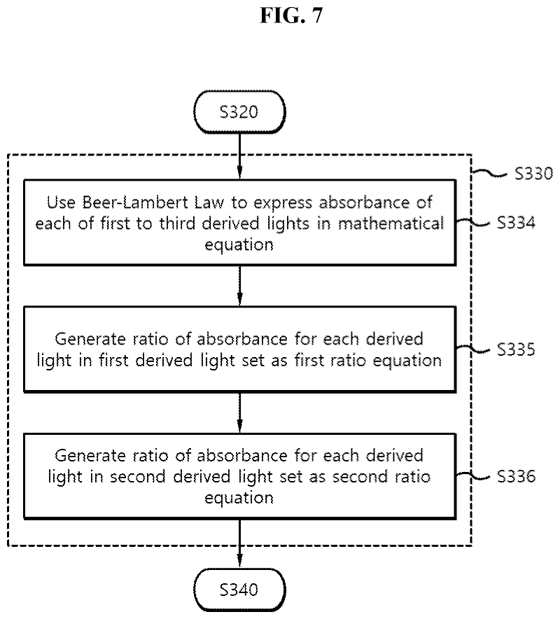

Hereinafter, stage S330 according to embodiments of the present disclosure will be described in more detail using FIGS. 6 and 7.

FIGS. 6 and 7 are flowcharts for explaining stage S330 illustrated in FIG. 3.

Hereinafter, a method for measurement of glycated hemoglobin according to the transmittance of LED light according to the first embodiment of the present disclosure will be described in more detail.

Referring to FIG. 6, the computation unit 130 may express each transmittance in a mathematical equation when the lights of the first LED module 111, the second LED module 112, and the third LED module 113 are radiated (stage S331).

According to the Photon-Diffusion Theory, the total absorption coefficient (<Cₐ) and the total reduced scattering coefficient (C′ₛ) may be expressed by Mathematical Equations 1 and 2 below.

$$[ Mathematical Equation 1 ]$$

$${C}_{a} = {V}_{a} {μ}_{a}^{art} ( λ ) + {V}_{v} {μ}_{a}^{vein} ( λ ) + {V}_{w} {μ}_{a}^{water} ( λ ) + [ 1 - ( {V}_{a} + {V}_{v} + {V}_{w} ) ] {μ}_{a}^{baseline}$$

In the mathematical equation above, Vₐ represents an arterial blood volume, Vᵥ represents a venous blood volume, V_{w} represents a water volume, μₐ^{art} represents an arterial blood absorption coefficient, μα^{vein} represents a venous blood absorption coefficient, μₐ^{water} represents a water absorption coefficient, and μₐ^{baseline} represents a reference tissue absorption coefficient.

$${C}_{s}^{′} = ( {V}_{a} + {V}_{v} ) {{μ}_{s}^{′}}^{blood} + [ 1 - ( {V}_{a} + {V}_{v} ) ] {μ}_{s}^{′ dermis} \qquad [ Mathematical Equation 2 ]$$

In the mathematical equation above, μ′ₛ^{blood} represents a reduced blood scattering coefficient, and μ′ₛ^{dermis} represents a reduced skin tissue scattering coefficient.

Blood is a homogeneous mixture and contains different types of hemoglobin: oxy-hemoglobin (HbO), deoxy-hemoglobin (HHb), and glycated hemoglobin (HbA1c).

Accordingly, the arterial blood absorption coefficient (μₐ^{art}) the venous blood absorption coefficient (μₐ^{vein}), and the reference tissue absorption coefficient (μₐ^{baseline}) in Mathematical Equation 1 may be expressed as Mathematical Equations 3 to 5, respectively, below.

$$[ Mathematical Equation 3 ]$$

$${μ}_{a}^{art} = {Sa O}_{2} {μ}_{a}^{HbO} + ( 1 - {Sa O}_{2} ) {μ}_{a}^{HHb} ) ( 1 - HbA 1 c ) + HbA 1 c {μ}_{a}^{{HbA 1 c}_{}}$$

In the mathematical equation above, SaO₂ represents arterial oxygen saturation, μₐ^{Hbo} represents an oxyhemoglobin absorption coefficient, μₐ^{HHb} represents a deoxy-hemoglobin absorption coefficient, and μₐ^{HbA1c} represents a glycated hemoglobin absorption coefficient.

$$[ Mathematical Equation 4 ]$$

$${μ}_{a}^{vein} = {Sv O}_{2} {μ}_{a}^{HbO} + ( 1 - {Sv O}_{2} ) {μ}_{a}^{HHb} ) ( 1 - HbA 1 c ) + HbA 1 c {μ}_{a}^{{HbA 1 c}_{}}$$

In the mathematical equation above, SvO₂ represents venous oxygen saturation.

$${μ}_{a}^{baseline} = 7.84 \times {10}^{7} \times {λ}^{- 3.255} \qquad [ Mathematical Equation 5 ]$$

Mathematical Equations 3 and 4 may be derived from Mathematical Equation 6 below.

$${SO}_{2} = \frac{{C}_{HbO}}{{C}_{HbO} + {C}_{HHb}} \qquad [ Mathematical Equation 6 ]$$

$$HbA 1 c = \frac{{C}_{HbA 1 c}}{{C}_{H bA 1 C} + {C}_{HbO} + {C}_{HHb}}$$

In the mathematical equation above, SO₂ includes arterial oxygen saturation SaO₂) and venous oxygen saturation (SvO₂). In addition, C_{HHb} represents the molar concentration the molar concentration of deoxy-hemoglobin, C_{HbO} represents the molar concentration of oxyhemoglobin, and C_{HbA1c} represents the molar concentration of glycated hemoglobin.

Next, the computation unit 130 may acquire a mathematical equation for transmittance using spherical geometry.

First, the photon diffusion equation based on spherical geometry may be expressed as Mathematical Equation 7 below.

$$\frac{1}{{ρ}^{2}} \frac{d}{d ρ} ( {ρ}^{2} \frac{d ψ ( ρ )}{d ρ} ) - {α}^{2} ψ ( ρ ) = - \frac{1}{D} S ( ρ ) \qquad [ Mathematical Equation 7 ]$$

In the mathematical equation above, ψ(ρ) represents the photon density with a scalar size in ρ, S(ρ) represents a source function, α represents a attenuation coefficient, and D represents a diffusion coefficient. In addition, ρ represents the distance between a power source (source) and an arbitrary point in a hemispherical space, as illustrated in FIG. 2.

In addition, the attenuation coefficient (α) and diffusion coefficient (D) in Mathematical Equation 7 may be simplified by the total absorption coefficient (Cₐ) and the total reduced scattering coefficient (C′ₛ) as shown in Mathematical Equation 8 below.

$$α = \sqrt{3 {C}_{a} ( {C}_{a} + {C}_{s}^{′} )} \approx \sqrt{3 {C}_{a} {C}_{s}^{′}} \qquad [ Mathematical Equation 8 ]$$

$$D = 1 / ( 3 ( {C}_{a} + {C}_{s}^{′} ) )$$

Assuming that the source function (S(ρ)) in Mathematical Equation 7 is Mathematical Equation 9 below, the photon density (ψ(ρ) may be converted to Mathematical Equation 10 below.

$$s ( ρ ) = \frac{3 {P}_{0}}{4 π} δ ( ρ ) \qquad [ Mathematical Equation 9 ]$$

In the mathematical equation above, P₀ represents the optical emission power emitted from the power source (source), and δ(ρ) represents the three-dimensional volumetric value of the two-dimensional Dirac delta function for the point source (power source).

$$ψ ( ρ ) = \frac{A}{p} {e}^{- αρ} + \frac{B}{p} {e}^{αp} \qquad [ Mathematical Equation 10 ]$$

In the mathematical equation above, coefficients A and B need to satisfy two boundary conditions, and the boundary conditions may be expressed by Mathematical Equation 11 below.

$$a ) {lim}_{ρ \to 0} [ flux density ] = {lim}_{ρ \to 0} [ - 4 π D {ρ}^{2} \frac{d ψ ( ρ )}{d ρ} ] = {P}_{0} \qquad [ Mathematical Equation 11 ]$$

$$b ) All photon flux is absorbed beyond the boundary ρ = d ψ ( ρ ) = 0 at ρ = d$$

In addition, when the boundary conditions according to Mathematical Equation 11 are satisfied, A and B may be converted to Mathematical Equation 12 below.

$$A = \frac{{P}_{0}}{4 π D} \frac{1}{1 - {e}^{- 2 α d}} \qquad [ Mathematical Equation 12 ]$$

$$B = \frac{{P}_{0}}{4 π D} \frac{1}{1 - {e}^{2 α d}}$$

In addition, the photon density (ψₛₚₕ(ρ) applied to spherical geometry may be expressed as Mathematical Equation 13 below.

$${ψ}_{sph} ( ρ ) = \frac{{P}_{0}}{4 π D} [ \frac{{C}_{1}}{ρ} {e}^{- αρ} + \frac{{C}_{2}}{P} {e}^{α ρ} ] \qquad [ Mathematical Equation 13 ]$$

In addition, when spherical geometry is applied to the system 100 for non-invasive measurement of glycated hemoglobin according to the first embodiment of the present disclosure, it may be expressed as Mathematical Equation 14 below.

Accordingly, as illustrated in FIG. 2, the computation unit 130 may acquire the flux density for the distance value (d) from the LED module 110 to the light detection unit 120 through Equation 14 below.

$${ψ}_{hemi} ( ρ ) = \frac{- 2 \partial z}{ρ} \frac{d {ψ}_{sph} ( ρ )}{d ρ} = \frac{\partial z {P}_{0}}{2 π D {ρ}^{2}} [ {C}_{1} {e}^{- λ ρ} ( \frac{1}{ρ} + α ) + {C}_{2} {e}^{αρ} ( \frac{1}{ρ} - α ) ] \qquad [ Mathematical Equation 14 ]$$

In the mathematical equation above, ψₕₑₘᵢ(ρ) represents the photon density according to the hemispherical geometry, ϑ represents a value indicating the depth below the surface where the first scattered photon is emitted, and z represents the orthogonal distance value from vector ρ to a source surface as shown in FIG. 2.

Next, the computation unit 130 may infer the intensity of the transmitted light (Iₜ) using Mathematical Equation 14 as shown in Mathematical Equation 15 below.

$${{I}_{t} = D \frac{\partial {ψ}_{hemi} ( ρ )}{\partial z} \vert}_{{ρ = d}_{r = 0}} = - \frac{\partial {P}_{0}}{2 π d} [ {C}_{1} {e}^{- α d} ( \frac{2}{{d}^{2}} + \frac{2 α}{d} + {α}^{2} ) + {C}_{2} {e}^{α d} ( \frac{2}{{d}^{2}} - \frac{2 α}{d} + {α}^{2} ) ] \qquad [ Mathematical Equation 15 ]$$

Accordingly, the transmitted light for each wavelength from the light detection unit 120 may be expressed by Mathematical Equation 16 below.

$$\frac{Δ {I}_{t}}{{I}_{t}} = \frac{- 3 {C}_{s}^{′} d}{2} {K}_{t} ( α , d ) {μ}_{a}^{art} Δ {V}_{a} \qquad [ Mathematical Equation 16 ]$$

$${K}_{t} ( α , d ) \approx \frac{α d - 1}{{α}^{2} d} for α d ≫ 1$$

In the mathematical equation above, Iₜ represents the intensity of transmitted light, Alt and represents the difference value of intensity of light between the peak value and valley value of the PPG signal.

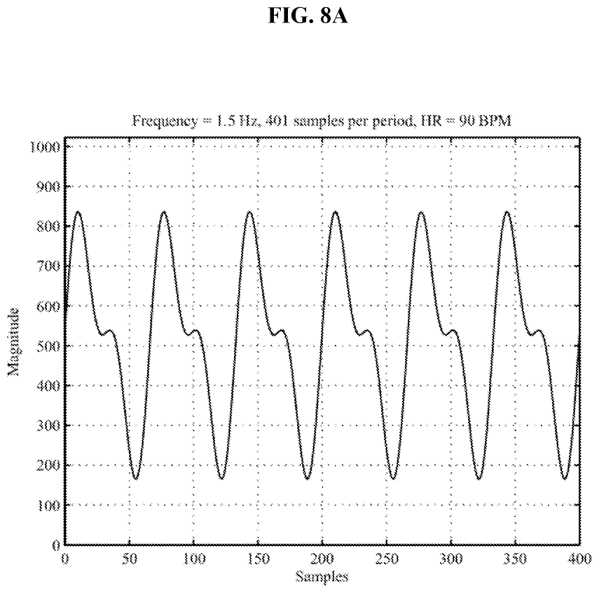

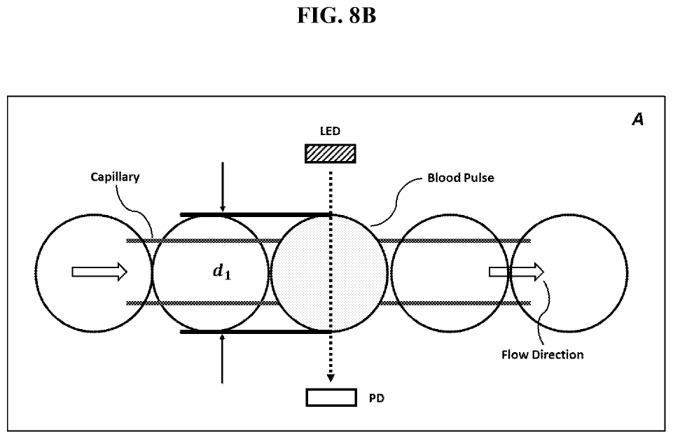

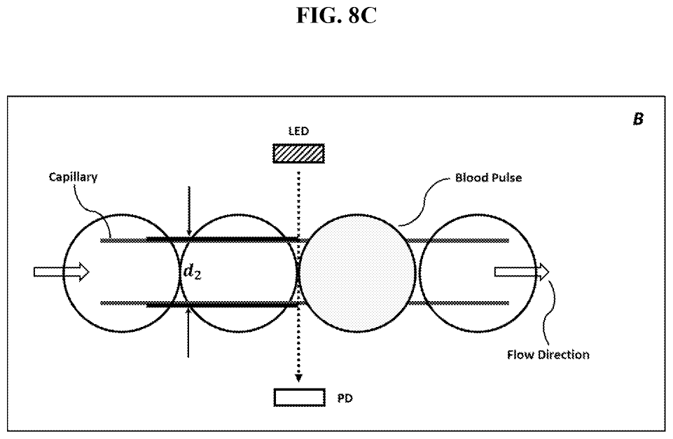

FIG. 8A is a graph showing PPG signals obtained by measuring photoblood flow in a portion of the body of a measurement subject.

FIG. 8B is a diagram for explaining a transmission distance (d) of LED light when blood enters capillaries. FIG. 8C is a diagram for explaining a transmission distance (d) of LED light when blood passes out the capillaries.

As shown in FIG. 8A, the size of the pulse value changes according to the flow of blood within the blood vessel, and the pulse size becomes maximum at the peak time (A) when blood enters the capillaries to the maximum. In this connection, as shown in FIG. 8b, the capillaries expand and the transmission distance (d) of the LED light increases.

As shown in FIG. 8A, the pulse size becomes minimum at the valley point (B) when blood flows out of the capillaries to the maximum. In this connection, as shown in FIG. 8C, the capillaries contract and the transmission distance (d) of the LED light decreases.

As such, when stage S331 is completed, the computation unit 130 may acquire a first ratio equation R1 representing the ratio of each transmittance when the lights of the second LED module 112 and the third LED module 113 are transmitted (stage S332).

In other words, the computation unit 130 may substitute a second wavelength (λ₂) and a third wavelength (λ₃) into the mathematical equation for transmittance acquired in stage S331 to acquire the first ratio equation R1 as shown in Mathematical Equation 17 below.

$${R}_{1} = \frac{{( \frac{Δ {I}_{t}}{{I}_{t}} )}_{λ 2}}{{( \frac{Δ {I}_{t}}{{I}_{t}} )}_{λ 3}} = \frac{{C}_{s}^{′} ( {λ}_{2} ) {K}_{t} ( {α}_{λ2} , d )}{{C}_{s}^{′} ( {λ}_{3} ) {K}_{t} ( {α}_{λ3} , d )} \cdot \frac{{μ}_{a}^{art} ( {λ}_{2} )}{{μ}_{a}^{art} ( {λ}_{3} )} \qquad [ Mathematical Equation 17 ]$$

In the mathematical equation above,

$${( \frac{Δ {I}_{t}}{{I}_{t}} )}_{λ 2}$$

represents the transmittance when a target is irradiated with the second LED having the second wavelength (λ₂), and

$${( \frac{Δ {I}_{t}}{{I}_{t}} )}_{λ 3}$$

represents the transmittance when a target is irradiated with the third LED having the third wavelength (λ₃).

Next, the computation unit 130 may acquire a second ratio equation R2 representing the ratio of each transmittance when the lights of the first LED module 111 and the third LED module 113 are transmitted (stage S333).

In other words, the computation unit 130 may substitute a first wavelength (λ₁) and a third wavelength (λ₃) into the mathematical equation for transmittance acquired in stage S331 to acquire the second ratio equation R2 as shown in Mathematical Equation 18 below.

$${R}_{2} = \frac{{( \frac{Δ {I}_{t}}{{I}_{t}} )}_{λ1}}{{( \frac{Δ {I}_{t}}{{I}_{t}} )}_{λ 3}} = \frac{{C}_{s}^{′} ( {λ}_{1} ) {K}_{t} ( {α}_{λ1} , d )}{{C}_{s}^{′} ( {λ}_{3} ) {K}_{t} ( {α}_{λ3} , d )} \cdot \frac{{μ}_{a}^{art} ( {λ}_{1} )}{{μ}_{a}^{art} ( {λ}_{3} )} \qquad [ Mathematical Equation 18 ]$$

In the mathematical equation above,

$${( \frac{Δ {I}_{t}}{{I}_{t}} )}_{λ1}$$

represents the transmittance when a target is irradiated with the second LED having the first wavelength (λ₁), and

$${( \frac{Δ {I}_{t}}{{I}_{t}} )}_{λ 3}$$

represents the transmittance when a target is irradiated with the third LED having the third wavelength (λ₃).

When stage S330 is completed, the computation unit 130 may apply the intensity of transmitted light of the first to third LED modules measured by the light detection unit 120 to the first ratio equation R1 and the second ratio equation R2, and compute the concentrations of glycated hemoglobin (HbA1c) and arterial blood oxygen saturation (SpO2) of the measurement subject (stage S340).

In addition, the first ratio equation R1 and the second ratio equation R2 acquired in Mathematical Equation 17 and Mathematical Equation 18 may be expressed as C′ₛ(λ)×Kₜ(α_{λ}, d)×μ_{A}^{art}(λ) in both the numerator and denominator. However, only the wavelength range may be different.

Accordingly, the first ratio equation R1 and the second ratio equation R2 may be simplified as shown in Mathematical Equations 19 and 20 below.

$$[ Mathematical Equation 19 ]$$

$${R}_{1} = \frac{\begin{matrix}{C}_{1} f ( {C}_{2} , {C}_{3} , {C}_{4} , {C}_{5} ) \\ f ( {C}_{6} , {C}_{7} , {C}_{8} , {C}_{9} ) \\ ( {C}_{10} h + ( h - 1 ) ( {C}_{11} s - {C}_{12} ) ) \\ ( 10 \\ \sqrt{f ( {C}_{13} , {C}_{14} , {C}_{15} , {C}_{16} ) f} \\ ( {C}_{17} , {C}_{18} , {C}_{19} , {C}_{20} ) - 1 )\end{matrix}}{\begin{matrix}f ( {C}_{21} , {C}_{22} , {C}_{23} , {C}_{24} ) \\ f ( {C}_{25} , {C}_{26} , {C}_{27} , {C}_{28} ) \\ ( {C}_{29} h + ( h - 1 ) ( {C}_{30} s - {C}_{31} ) ) \\ ( 10 \\ \sqrt{f ( {C}_{32} , {C}_{33} , {C}_{34} , {C}_{35} ) f} \\ ( {C}_{36} , {C}_{37} , {C}_{38} , {C}_{39} ) - 1 )\end{matrix}}$$

$$f ( a , b , c , d ) = ahs + bh + cs + d$$

$$[ Mathematical Equation 20 ]$$

$${R}_{2} = \frac{\begin{matrix}{C}_{1} f ( {C}_{2} , {C}_{3} , {C}_{4} , {C}_{5} ) \\ f ( {C}_{6} , {C}_{7} , {C}_{8} , {C}_{9} ) \\ ( {C}_{10} h + ( h - 1 ) ( {C}_{11} s - {C}_{12} ) ) \\ ( 10 \\ \sqrt{f ( {C}_{13} , {C}_{14} , {C}_{15} , {C}_{16} ) f} \\ ( {C}_{17} , {C}_{18} , {C}_{19} , {C}_{20} ) - 1 )\end{matrix}}{\begin{matrix}f ( {C}_{21} , {C}_{22} , {C}_{23} , {C}_{24} ) \\ f ( {C}_{25} , {C}_{26} , {C}_{27} , {C}_{28} ) \\ ( {C}_{29} h + ( h - 1 ) ( {C}_{30} s - {C}_{31} ) ) \\ ( 10 \\ \sqrt{f ( {C}_{32} , {C}_{33} , {C}_{34} , {C}_{35} ) f} \\ ( {C}_{36} , {C}_{37} , {C}_{38} , {C}_{39} ) - 1 )\end{matrix}}$$

$$f ( a , b , c , d ) = ahs + bh + cs + d$$

In the mathematical equation above, C₁to C₃₉ represent coefficient values used in the ratio equations R1 and R2, h represents glycated hemoglobin, and s represents oxygen saturation.

For example, it is assumed that the first wavelength (λ₁) value is 525 nm, the second wavelength (λ₂) value is 660 nm, and the third wavelength (λ₃) value is 950 nm.

In addition, C₁ to C₃₉ applied to the first ratio equation R1 using the second wavelength (λ₂) and the third wavelength (λ₃) may be acquired as shown in Table 1 below.

TABLE 1
R1 - 660 nm-950 nm

| C1 | C2 | C3 | C4 | C5 | C6 | C7 | C8 | C9 | C10 |
|---|---|---|---|---|---|---|---|---|---|
| 1.44 | −7.22 | 1983.22 | 7.22 | 14.73 | −2.41 | 661.07 | 2.41 | 24.15 | 81438 |
| C11 | C12 | C13 | C14 | C15 | C16 | C17 | C18 | C19 | C20 |
| 2907.45 | 3226.9 | 11.63 | 312.84 | −11.63 | 40.74 | 34.89 | 938.53 | −34.89 | 38.85 |
| C21 | C22 | C23 | C24 | C25 | C26 | C27 | C28 | C29 | C30 |
| 11.63 | 312.85 | −11.63 | 40.74 | 34.89 | 938.53 | −34.89 | 38.85 | 165870 | −601.92 |
| C31 | C32 | C33 | C34 | C35 | C36 | C37 | C38 | C39 | |
| 602.08 | −7.22 | 1983.22 | 7.22 | 14.73 | −2.41 | 661.07 | 2.41 | 24.15 | |

In addition, C₁to C₃₉ applied to the second ratio equation R2 using the first wavelength (λ₁) and the third wavelength (λ₃) may be acquired as shown in Table 2 below.

TABLE 2
R2 - 525 nm-950 nm

| C1 | C2 | C3 | C4 | C5 | C6 | C7 | C8 | C9 | C10 |
|---|---|---|---|---|---|---|---|---|---|
| 2.06 | −7.22 | 1983.22 | 7.22 | 14.73 | −2.41 | 661.07 | 2.41 | 24.15 | 589160 |
| C11 | C12 | C13 | C14 | C15 | C16 | C17 | C18 | C19 | C20 |
| 4335 | 35157 | 17.34 | 2216.01 | −17.34 | 180.26 | 52.02 | 6648.04 | −52.02 | 422.01 |
| C21 | C22 | C23 | C24 | C25 | C26 | C27 | C28 | C29 | C30 |
| 17.34 | 2216.01 | −17.34 | 180.26 | 52.02 | 6648.04 | −52.02 | 422.01 | 165870 | −601.92 |
| C31 | C32 | C33 | C34 | C35 | C36 | C37 | C38 | C39 | |
| 602.08 | −7.22 | 1983.22 | 7.22 | 14.73 | −2.41 | 661.07 | 2.41 | 24.15 | |

Next, the computation unit 130 may apply the first ratio equation R1 and the second ratio equation R2 to the function (f) of Mathematical Equation 21 below to calculate the concentrations of glycated hemoglobin (HbA1c) and arterial blood oxygen saturation (SpO2).

$$[ Mathematical Equation 21 ]$$

$$s = f ( {R}_{1} , {R}_{2} )$$

$$h = f ( {R}_{1} , {R}_{2} )$$

In the mathematical equation above, h and s represent the concentration values of glycated hemoglobin (HbA1c) and oxygen saturation (SpO2) to be measured in blood, respectively.

In other words, s=% SpO2/100 and h=% HbA1C/100.

Accordingly, according to the first embodiment of the present disclosure, by acquiring the intensity of light of three LEDs measured by the light detection unit 120 installed on the body of the measurement subject and applying the ratios R1 and R2 of each acquired intensity value of light to the generated Mathematical Equation 21, the concentration of glycated hemoglobin (HbA1c) and the concentration of arterial blood oxygen saturation (SpO2) may be calculated. In addition, the calculated concentration of glycated hemoglobin (HbA1c) may be used to estimate blood sugar level.

Hereinafter, the method for measuring glycated hemoglobin according to reflectance of LED light according to the second embodiment of the present disclosure will be described in more detail.

Referring to FIG. 6, the computation unit 130 may express each reflectance when the lights of the first LED module 111, the second LED module 112, and the third LED module 113 are radiated using a mathematical equation (stage S331).

The computation unit 130 may compute the total absorption coefficient (Cₐ) and the total reduced scattering coefficient (C′ₛ) according to the Photon-Diffusion Theory using Mathematical Equations 1 to 6 described above.

Next, the computation unit 130 may acquire the flux density for the distance value (r) from the plurality of LED modules 110 to the light detection unit 120 using Mathematical Equations 7 to 13.

The Photon-Diffusion Theory and the theorem on spherical geometry are well-known techniques that may be easily implemented by those skilled in the art, and are equally applied to the first and second embodiments. Therefore, in the second embodiment of the present disclosure, the redundant description of Mathematical Equations 7 to 13 is omitted.

Next, the computation unit 130 may infer the intensity of the transmitted light (Iᵣ) using Mathematical Equation 14 as shown in Mathematical Equation 22 below.

$$[ Mathematical Equation 22 ]$$

$${{I}_{r} = - D \frac{\partial {ψ}_{hemi} ( ρ )}{\partial z} \vert}_{{ρ = r}_{z = 0}} = - \frac{ϑ {P}_{0}}{2 π d} [ {C}_{1} {e}^{- α d} ( \frac{1}{r} + α ) + {C}_{2} {e}^{α d} ( \frac{1}{r} - α ) ]$$

In the mathematical equation above, r represents the distance value between the plurality of LED modules 110 and the light detection unit 120, as illustrated in FIG. 2.

In addition, the reflected light for each wavelength detected from the light detection unit 120 may be expressed as Mathematical Equation 23 below.

$$[ Mathematical Equation 23 ]$$

$$\frac{Δ {I}_{r}}{{I}_{r}} = \frac{3 {C}_{s}^{′}}{2} {K}_{r} ( α , r ) {μ}_{a}^{art} Δ {V}_{a}$$

$${K}_{r} ( α , r ) = \frac{- {r}^{2}}{1 + α r}$$

In the mathematical equation above, Iᵣ represents the intensity of diffused light, and ΔIᵣ represents the difference value of intensity of light between the peak value and valley value of the PPG signal.

The difference between the peak value and valley value of the PPG signal may be explained with reference to FIGS. 8A to 8C as in the first embodiment.

When stage S331 is completed, the computation unit 130 may acquire a first ratio equation R1 representing the ratio of each reflectance when the lights of the second LED module 112 and the third LED module 113 are transmitted (stage S332).

In other words, the computation unit 130 may substitute the second wavelength (λ₂) and the third wavelength (λ₃) into the mathematical equation for reflectance acquired in stage S331 to acquire the first ratio equation R1 as shown in Mathematical Equation 24 below.

$$[ Mathematical Equation 24 ]$$

$${R}_{1} = \frac{{( \frac{Δ {I}_{r}}{{I}_{r}} )}_{λ2}}{{( \frac{Δ {I}_{r}}{{I}_{r}} )}_{λ3}} = \frac{{C}_{s}^{′} ( {λ}_{2} ) {K}_{r} ( {α}_{λ2} , r )}{{C}_{s}^{′} ( {λ}_{3} ) {K}_{r} ( {α}_{λ3} , r )} \cdot \frac{{μ}_{a}^{art} ( {λ}_{2} )}{{μ}_{a}^{art} ( {λ}_{3} )}$$

In the mathematical equation above,

$${( \frac{Δ {I}_{r}}{{I}_{r}} )}_{λ2}$$

represents the reflectance when the second LED having the second wavelength (λ₂) is radiated, and

$${( \frac{Δ {I}_{r}}{{I}_{r}} )}_{λ3}$$

represents the reflectance when the third LED having the third wavelength (λ₃) is radiated.

Next, the computation unit 130 may acquire a second ratio equation R2 representing the ratio of each reflectance when the lights of the first LED module 111 and the third LED module 113 are radiated (stage S333).

In other words, the computation unit 130 may substitute the first wavelength (λ₁) and the third wavelength (λ₃) into the mathematical equation for reflectance acquired in stage S331 to acquire the second ratio equation R2 as shown in Mathematical Equation 25 below.

$$[ Mathematical Equation 25 ]$$

$${R}_{2} = \frac{{( \frac{Δ {I}_{r}}{{I}_{r}} )}_{λ1}}{{( \frac{Δ {I}_{r}}{{I}_{r}} )}_{λ3}} = \frac{{C}_{s}^{′} ( {λ}_{1} ) {K}_{r} ( {α}_{λ1} , r )}{{C}_{s}^{′} ( {λ}_{3} ) {K}_{r} ( {α}_{λ3} , r )} \cdot \frac{{μ}_{a}^{art} ( {λ}_{1} )}{{μ}_{a}^{art} ( {λ}_{3} )}$$

In the mathematical equation above,

$${( \frac{Δ {I}_{r}}{{I}_{r}} )}_{λ1}$$

represents the reflectance when the first LED having the first wavelength (λ₁) is radiated, and

$${( \frac{Δ {I}_{r}}{{I}_{r}} )}_{λ3}$$

represents the reflectance when the third LED having the third wavelength (λ₃) is radiated.

When stage S330 is completed, the computation unit 130 may apply the intensity of reflected light of the first to third LEDs measured by the light detection unit 120 to the first ratio equation R1 and the second ratio equation R2, and compute the concentrations of glycated hemoglobin (HbA1c) and arterial blood oxygen saturation (SpO2) of the measurement subject (stage S340).

In addition, the first ratio equation R1 and the second ratio equation R2 acquired in Mathematical Equation 24 and Mathematical Equation 25 may be expressed as C′ₛ(λ)×Kᵣ(α_{λ},r)×μ_{A}^{art}(λ) in both the numerator and denominator. However, only the wavelength range may be different.

Accordingly, the first ratio equation R1 and the second ratio equation R2 acquired in Mathematical Equation 24 and Mathematical Equation 25 may be simplified and expressed as Mathematical Equation 26 and Mathematical Equation 27 below.

$$[ Mathematical Equation 26 ]$$

$${R}_{1} = \frac{\begin{matrix}{C}_{1} ( {C}_{2} h + ( h - 1 ) ( {C}_{3} s + {C}_{4} ) ) ( 2 \\ \sqrt{f ( {C}_{5} , {C}_{6} , {C}_{7} , {C}_{8} ) f} \\ ( {C}_{9} , {C}_{10} , {C}_{11} , {C}_{12} ) + 1 )\end{matrix}}{\begin{matrix}( {C}_{13} h + ( h - 1 ) ( {C}_{14} s + {C}_{15} ) ) ( 2 \\ \sqrt{f ( {C}_{16} , {C}_{17} , {C}_{18} , {C}_{19} ) f} \\ ( {C}_{20} , {C}_{21} , {C}_{22} , {C}_{23} ) + 1 )\end{matrix}}$$

$$f ( a , b , c , d ) = ahs + bh + cs + d$$

$$[ Mathematical Equation 27 ]$$

$${R}_{2} = \frac{\begin{matrix}{C}_{1} ( {C}_{2} h + ( h - 1 ) ( {C}_{3} s + {C}_{4} ) ) ( 2 \\ \sqrt{f ( {C}_{5} , {C}_{6} , {C}_{7} , {C}_{8} ) f} \\ ( {C}_{9} , {C}_{10} , {C}_{11} , {C}_{12} ) + 1 )\end{matrix}}{\begin{matrix}( {C}_{13} h + ( h - 1 ) ( {C}_{14} s + {C}_{15} ) ) ( 2 \\ \sqrt{f ( {C}_{16} , {C}_{17} , {C}_{18} , {C}_{19} ) f} \\ ( {C}_{20} , {C}_{21} , {C}_{22} , {C}_{23} ) + 1 )\end{matrix}}$$

$$f ( a , b , c , d ) = ahs + bh + cs + d$$

Here, C₁ to C₂₃ represent coefficient values used in the ratio equations R1 and R2), h represents glycated hemoglobin, and s represents oxygen saturation.

For example, it is assumed that the first wavelength (λ₁) value is 525 nm, the second wavelength (λ₂) value is 660 nm, and the third wavelength ((λ₃) value is 950 nm.

In addition, C₁ to C₂₃ applied to the first ratio equation R1 using the second wavelength (λ₂) and the third wavelength (λ₃) may be acquired as shown in Table 3 below.

TABLE 3
R1 - 660 nm-950 nm

| C1 | C2 | C3 | C4 | C5 | C6 | C7 | C8 | C9 | C10 |
|---|---|---|---|---|---|---|---|---|---|
| 1.44 | 81438 | 2907.45 | −3226.9 | −7.22 | 1983.22 | 7.22 | 14.73 | −2.41 | 661.07 |
| C11 | C12 | C13 | C14 | C15 | C16 | C17 | C18 | C19 | C20 |
| 2.41 | 24.15 | 165870 | 601.92 | 602.08 | 11.63 | 312.84 | −11.63 | 40.74 | 34.89 |
| C21 | C22 | C23 | | | | | | | |
| 938.53 | −34.89 | 38.85 | | | | | | | |

In addition, C₁ to C₂₃ applied to the second ratio equation R2 using the first wavelength (λ₁) and the third wavelength (λ₃) may be acquired as shown in Table 4 below.

TABLE 4
R2 - 525 nm-950 nm

| C1 | C2 | C3 | C4 | C5 | C6 | C7 | C8 | C9 | C10 |
|---|---|---|---|---|---|---|---|---|---|
| 2.06 | 589160 | 4335 | −35157 | −7.22 | 1983.22 | 7.22 | 14.73 | −2.41 | 661.07 |
| C11 | C12 | C13 | C14 | C15 | C16 | C17 | C18 | C19 | C20 |
| 2.41 | 24.15 | 165870 | 601.92 | 602.08 | 17.34 | 2216.01 | −17.34 | 180.26 | 52.02 |
| C21 | C22 | C23 | | | | | | | |
| 6648.04 | −52.02 | 422.01 | | | | | | | |

Next, the computation unit 130 may apply the first ratio equation R1 and the second ratio equation R2 to the function (f) of Mathematical Equation 28 below to calculate the concentrations of glycated hemoglobin (HbA1c) and arterial blood oxygen saturation (SpO2).

$$[ Mathematical Equation 28 ]$$

$$s = f ( {R}_{1} , {R}_{2} )$$

$$h = f ( {R}_{1} , {R}_{2} )$$

In the mathematical equation above, h and s represent the concentration values of glycated hemoglobin (HbA1c) and oxygen saturation (SpO2) to be measured in blood, respectively.

In other words, s=% SpO2/100 and h=% HbA1C/100.

In the first and second embodiments of the present disclosure, when two LEDs having different wavelengths are transmitted, the ratio of each transmittance or reflectance is generated using a mathematical equation, and three LEDs are applied to calculate the concentration of unknown, namely glycated hemoglobin (HbA1c) and arterial blood oxygen saturation (SpO2), using the ratio equation for the generated transmittance or reflectance, without being limited thereto. To increase the accuracy of detecting the concentrations of glycated hemoglobin (HbA1c) and arterial blood oxygen saturation (SpO2), the number of LEDs may be added and installed.

According to the method for measurement of glycated hemoglobin using the system for measurement of glycated hemoglobin according to the second embodiment of the present disclosure, accurate measurement is possible no matter where the plurality of LED modules 110 and the light detection unit 120 are attached to any point on the body. Accordingly, it is possible to accurately measure the concentrations of glycated hemoglobin (HbA1c) and arterial blood oxygen saturation (SpO2) without being affected by expansion of capillaries due to changes in surrounding temperature.

As such, according to an embodiment of the present disclosure, the concentration of glycated hemoglobin (HbA1C) is measured non-invasively using the intensity change rate of LED light having three different wavelengths. According to the Photon-Diffusion Theory, the concentrations of glycated hemoglobin (HbA1c) and arterial blood oxygen saturation (SpO2) may be measured accurately and easily through the intensity change rate of light to which the transmittance of LED light is applied or the intensity change rate of light to which the reflectance of LED light is applied.

Hereinafter, a method for measuring glycated hemoglobin according to the absorbance of LED light according to the third embodiment of the present disclosure will be described in more detail.

Referring to FIG. 7, the computation unit 130 may use Beer-Lambert Law to express the absorbance of each of the first LED module 111, the second LED module 112, and the third LED module 113 in a mathematical equation when the light is transmitted therethrough (stage S334).

Herein, the Beer-Lambert Law may be expressed as Mathematical Equation 29 below.

$$[ Mathematical Equation 29 ]$$

$$A = {\sum }_{i = 1}^{N} {A}_{i} = {\sum }_{i = 1}^{N} {ϵ}_{i} \times {c}_{i} \times d = - ln ( \frac{I}{{I}_{0}} )$$

In the mathematical equation above, A represents the absorbance, N represents the number of types of hemoglobin, ϵ represents the molar extinction coefficient, c represents the molar concentration of the object through which light is transmitted, d represents the transmission distance of light, I₀ represents the intensity of incident light, and I represents the intensity of light detected after transmission.

Blood is a homogeneous mixture and contains different types of hemoglobin. In an embodiment of the present disclosure, the absorbance of oxy-hemoglobin (HbO), deoxy-hemoglobin (HHb), and glycated hemoglobin (HbA1c) may be calculated.

Accordingly, the computation unit 130 may express the absorbance (A) when the LED is transmitted through the homogeneous mixture using Mathematical Equation 30 below.

$$[ Mathematical Equation 30 ]$$

$$\begin{matrix}A = {ϵ}_{HHb} ( λ ) \times {c}_{HHb} \times d + {ϵ}_{HbO} ( λ ) \times {c}_{HbO} \times d + {ϵ}_{HbA 1 c} ( λ ) \times {c}_{HbA 1 c} \times d \\ = ( {ϵ}_{HHb} ( λ ) \times {c}_{HHb} + {ϵ}_{HbO} ( λ ) \times {c}_{HbO} + {ϵ}_{HbA 1 c} ( λ ) \times {c}_{HbA 1 c} ) \times d\end{matrix}$$

In the mathematical equation above, ϵ_{HHb} represents the molar extinction coefficient of deoxy-hemoglobin, C_{HHb} represents the molar concentration of deoxy-hemoglobin, ϵ_{HbO} represents the molar extinction coefficient of oxy-hemoglobin, C_{HbO} represents the molar concentration of oxy-hemoglobin, ϵ_{HbA1c} represents the molar extinction coefficient of glycated hemoglobin, C_{HbA1c} represents the molar concentration of glycated hemoglobin, and d represents the transmission distance.

Since the width of the capillaries repeatedly expands and contracts according to the inflow of blood, the transmission distance (d) of the LED light varies depending on the inflow of blood. Accordingly, the computation unit 130 may substitute the changing transmission distance value and express the same in Mathematical Equation 31 below.

$$[ Mathmatical Equation 31 ]$$

$$δ A = ( {ϵ}_{HHb} ( λ ) \times {c}_{HHb} + {ϵ}_{HbO} ( λ ) \times {c}_{HbO} + {ϵ}_{HbA 1 c} ( λ ) \times {c}_{HbA 1 c} ) \times δ d$$

In the mathematical equation above, δd represents the difference value between d₁ and d₂, where d₁ represents the transmission distance of light when blood enters the capillaries, and d₂ represents the transmission distance of light when blood flows out of the capillaries to the surroundings.

That is, considering that when blood flows into and out of capillaries, the blood vessels repeat expansion and contraction, and thus the thickness of the fingers, etc. changes slightly, δd may be expressed as the difference value between d₁ and d₂.

When stage S334 is completed, the computation unit 130 may acquire a first equation R1 representing the ratio of each absorbance when the lights of the second LED module 112 and the third LED module 113 are transmitted (stage S335).

In other words, the computation unit 130 may substitute a second wavelength (λ₂), and a third wavelength (λ₃) into the mathematical equation for absorbance acquired in stage S334 to acquire the first equation R1 as shown in Mathematical Equation 32 below.

$${R}_{1} = \frac{δ {A}_{λ2}}{δ {A}_{λ3}} = \frac{\begin{matrix}{ϵ}_{HHb} ( {λ}_{2} ) \times {c}_{HHb} + \\ {ϵ}_{HbO} ( {λ}_{2} ) \times {c}_{HbO} + {ϵ}_{HbA 1 c} ( {λ}_{2} ) \times {c}_{HbA 1 c}\end{matrix}}{\begin{matrix}{ϵ}_{HHb} ( {λ}_{3} ) \times {c}_{HHb} + \\ {ϵ}_{HbO} ( {λ}_{3} ) \times {c}_{HbO} + {ϵ}_{HbA 1 c} ( {λ}_{3} ) \times {c}_{HbA 1 c}\end{matrix}} \qquad [ Mathematical Equation 32 ]$$

In the mathematical equation above, δA_{λ2} represents the absorbance corresponding to δd when a target is irradiated with the second LED having the second wavelength (λ₂), in other words, the difference between the absorbance at d₁ and the absorbance at d₂, and δA_{λ3} represents the difference between the absorbance at d₁ and the absorbance at d₂ when a target is irradiated with the third LED having the third wavelength (λ₃).

Next, the computation unit 130 may acquire a second equation R2 representing the ratio of each absorbance when the lights of the first LED module 111 and the third LED module 113 are transmitted (stage S336).

In other words, the computation unit 130 may substitute a first wavelength (λ₁) and a third wavelength (λ₃) into the mathematical equation for absorbance acquired in stage S334 to acquire the second equation R2 as shown in Mathematical Equation 33 below.

$${R}_{2} = \frac{δ {A}_{λ1}}{δ {A}_{λ3}} = \frac{\begin{matrix}{ϵ}_{HHb} ( {λ}_{1} ) \times {c}_{HHb} + \\ {ϵ}_{HbO} ( {λ}_{1} ) \times {c}_{HbO} + {ϵ}_{HbA 1 c} ( {λ}_{1} ) \times {c}_{HbA 1 c}\end{matrix}}{\begin{matrix}{ϵ}_{HHb} ( {λ}_{3} ) \times {c}_{HHb} + \\ {ϵ}_{HbO} ( {λ}_{3} ) \times {c}_{HbO} + {ϵ}_{HbA 1 c} ( {λ}_{3} ) \times {c}_{HbA 1 c}\end{matrix}} \qquad [ Mathematical Equation 33 ]$$

In the mathematical equation above, δA_{λ1} represents the difference between the absorbance at d₁ and the absorbance at d₂ when a target is irradiated with the first LED having the first wavelength (λ₁) and δA_{λ3} represents the difference between the absorbance at d₁ and the absorbance at d₂ when a target is irradiated with the third LED having the third wavelength (λ₃).

When stages S335 and S336 are completed, the computation unit 130 applies the percentage of glycated hemoglobin (HbA1c) and the percentage of arterial blood oxidation saturation (SpO2) previously defined in the acquired first equation R1 and second equation R2 (stage S340).

Herein, the percentage of glycated hemoglobin (HbA1c) and the percentage of arterial blood oxidation saturation (SpO2) may be expressed as Mathematical Equation 34 below.

$$[ Mathematical Equation 34 ]$$

$$\% HbA 1 c = \frac{{c}_{HbA 1 c}}{{c}_{HHb} + {c}_{HbO} + {c}_{HbA 1 c}} \times 100 \%$$

$$\% SpO 2 = \frac{{c}_{HbO}}{{c}_{HHb} + {c}_{HbO}} \times 100 \%$$

In the mathematical equation above, C_{HHb} represents the molar concentration of deoxyhemoglobin, C_{HbO} represents the molar concentration of oxyhemoglobin, and C_{HbA1c} represents the molar concentration of glycated hemoglobin.

In addition, the computation unit 130 may convert the predefined percentage of glycated hemoglobin (HbA1c) and the predefined percentage of arterial blood oxidation saturation (SpO2) into the molar concentration of oxyhemoglobin C_{HbO}, the molar concentration of deoxyhemoglobin C_{HHb}, and the molar concentration of glycated hemoglobin C_{HbA1c}, respectively, as shown in Mathematical Equation 35 below.

$${c}_{HbO} = \frac{\% SpO 2}{100} \times ( {c}_{HHb} + {c}_{HbO} ) \qquad [ Mathematical Equation 35 ]$$

$${c}_{HHb} = ( 1 - \frac{\% SpO 2}{100} ) \times ( {c}_{HHb} + {c}_{HbO} )$$

$${c}_{HbA 1 c} = \frac{\% HbA 1 c}{100} \times ( {c}_{HHb} + {c}_{HbO} + {c}_{HbA 1 c} )$$

Next, the computation unit 130 may develop the molar concentration of oxidized hemoglobin C_{HbO} and the molar concentration of deoxyhemoglobin C_{HHb} as shown in Mathematical Equation 36 below.

$${c}_{HHb} + {c}_{HbO} = ( 1 - \frac{\% HbA 1 c}{100} ) \times ( {c}_{HHb} + {c}_{HbO} + {c}_{HbA 1 c} ) \qquad [ Mathematical Equation 36 ]$$

In addition, the computation unit 130 may use the generated Mathematical Equation 36 to convert the molar concentration of oxidized hemoglobin C_{HbO} and the molar concentration of deoxyhemoglobin C_{HHb} as shown in Mathematical Equation 37.

$${c}_{HbO} = \frac{\% SpO 2}{100} \times ( 1 - \frac{\% HbA 1 c}{100} ) \times ( {c}_{HHb} + {c}_{HbO} + {c}_{HbA 1 c} ) \qquad [ Mathematical Equation 37 ]$$

$${c}_{HHb} = ( 1 - \frac{\% SpO 2}{100} ) \times ( 1 - \frac{\% HbA 1 c}{100} ) \times ( {c}_{HHb} + {c}_{HbO} + {c}_{HbA 1 c} )$$

Next, the computation unit 130 may apply each of the molar concentration of oxidized hemoglobin C_{HbO}, the molar concentration of deoxyhemoglobin C_{HHb}, and the molar concentration of glycated hemoglobin C_{HbA1c} converted to the first equation R1 and the second equation R2, and may convert the same as shown in Mathematical Equation 38 below.

$${R}_{1} = \frac{\begin{matrix}{ϵ}_{HHb} ( {λ}_{2} ) \times \\ ( 1 - SpO 2 ) \times ( 1 - HbA 1 c ) + \\ {ϵ}_{HbO} ( {λ}_{2} ) \times SpO 2 \times ( 1 - HbA 1 c ) + \\ {ϵ}_{HbA 1 c} ( {λ}_{2} ) \times HbA 1 c\end{matrix}}{\begin{matrix}{ϵ}_{HHb} ( {λ}_{3} ) \times \\ ( 1 - SpO 2 ) \times ( 1 - HbA 1 c ) + \\ {ϵ}_{HbO} ( {λ}_{3} ) \times SpO 2 \times ( 1 - HbA 1 c ) + \\ {ϵ}_{HbA 1 c} ( {λ}_{3} ) \times HbA 1 c\end{matrix}} \qquad [ Mathematical Equation 38 ]$$

$${R}_{2} = \frac{\begin{matrix}{ϵ}_{HHb} ( {λ}_{1} ) \times ( 1 - SpO 2 ) \times ( 1 - HbA 1 c ) + \\ {ϵ}_{HbO} ( {λ}_{1} ) \times SpO 2 \times ( 1 - HbA 1 c ) + {ϵ}_{HbA 1 c} ( {λ}_{1} ) \times HbA 1 c\end{matrix}}{\begin{matrix}{ϵ}_{HHb} ( {λ}_{3} ) \times ( 1 - SpO 2 ) \times ( 1 - HbA 1 c ) + \\ {ϵ}_{HbO} ( {λ}_{3} ) \times SpO 2 \times ( 1 - HbA 1 c ) + {ϵ}_{HbA 1 c} ( {λ}_{3} ) \times HbA 1 c\end{matrix}}$$

In the mathematical equation above, it is defined as

$$SpO 2 = \frac{\% SpO 2}{100} , and$$

$$HbA 1 c = \frac{\% HbA 1 c}{100} .$$

In addition, ϵ_{HHb}(λ₁), ϵ_{HHb}(λ₂), and ϵ_{HHb}(λ₃) represent the molar extinction coefficients of deoxyhemoglobin when the first, second, and third wavelengths are applied, respectively; ϵ_{HbO}(λ₁), ϵ_{HbO}(λ₂), ϵ_{HbO}(λ₃) , and represent the molar extinction coefficients of oxidized hemoglobin when the first, second, and third wavelengths are applied, respectively; and ϵ_{HBA1c}(λ₁), ϵ_{HBA1c}(λ₂), and ϵ_{HBA1c}(λ₃) represent the molar extinction coefficients of glycated hemoglobin when the first, second, and third wavelengths are applied, respectively.

When stage S330 is completed, the computation unit 130 may apply the intensity of the incident light and the intensity of the light measured by the light detection unit 120 to the first equation R1 and the second equation R2 to compute the concentrations of the glycated hemoglobin (HbA1c) and the arterial blood oxidation saturation (SpO2) of the measurement subject (stage S340).

In addition, the first equation R1 and the second equation R2 may be expressed as mathematical equation 39 below according to the Beer-Lambert Law.

$$[ Mathematical Equation 39 ]$$

$${R}_{1} = \frac{δ {A}_{λ2}}{δ {A}_{λ3}} = \frac{{[ ln \frac{I ( {d}_{1} )}{I ( {d}_{2} )} ]}_{λ2}}{{[ ln \frac{I ( {d}_{1} )}{I ( {d}_{2} )} ]}_{λ3}} , {R}_{2} = \frac{δ {A}_{λ1}}{δ {A}_{λ3}} = \frac{{[ ln \frac{I ( {d}_{1} )}{I ( {d}_{2} )} ]}_{λ1}}{{[ ln \frac{I ( {d}_{1} )}{I ( {d}_{2} )} ]}_{λ3}}$$

In the mathematical equation above, δA_{λ1} represents the absorbance when a target is irradiated with the first LED having the first wavelength (λ₁), δA1_{80 2} represents the absorbance when a target is irradiated with the second LED having the second wavelength (λ₂), δA_{λ3} represents the absorbance when a target is irradiated with the third LED having the third wavelength (λ₃), d₁ and d₂ represent the light transmission distance when blood enters and exits, respectively, and I(d₁) and I(d₂) represent the intensity of light corresponding to d₁ and d₂, respectively.

When the first equation R1 in Mathematical Equation 39 is developed, it may be expressed as Mathematical Equation 40 below.

$$[ Mathematical Equation 40 ]$$

$${R}_{1} = \frac{δ {A}_{λ2}}{δ {A}_{λ3}} = \frac{{δ [ ln \frac{I}{{I}_{0}} ]}_{λ2}}{{δ [ ln \frac{I}{{I}_{0}} ]}_{λ3}} = \frac{{[ ln \frac{I ( {d}_{1} )}{{I}_{0} ( {d}_{1} )} - ln \frac{I ( {d}_{2} )}{{I}_{0} ( {d}_{2} )} ]}_{λ2}}{{[ ln \frac{I ( {d}_{1} )}{{I}_{0} ( {d}_{1} )} - ln \frac{I ( {d}_{2} )}{{I}_{0} ( {d}_{2} )} ]}_{λ3}} = \frac{{[ ln \frac{I ( {d}_{1} )}{I ( {d}_{2} )} ]}_{λ2}}{{[ ln \frac{I ( {d}_{1} )}{I ( {d}_{2} )} ]}_{λ3}}$$

In other words, I(d₁) represents the light intensity of the LED when the transmission distance is d₁, and I(d₂) represents the light intensity of the LED when the transmission distance is d₂. Accordingly, the first equation R1 may be expressed as a formula for the second) wavelength (λ₂) and the third wavelength (λ₃).

As such, the second equation R2 shown in Equation 39 may be developed and expressed as Mathematical Equation 41 below.

$${R}_{2} = \frac{δ {A}_{λ1}}{δ {A}_{λ3}} = \frac{{δ [ ln \frac{I}{{I}_{0}} ]}_{λ1}}{{δ [ ln \frac{I}{{I}_{0}} ]}_{λ3}} = \frac{{[ ln \frac{I ( {d}_{1} )}{{I}_{0} ( {d}_{1} )} - ln \frac{I ( {d}_{2} )}{{I}_{0} ( {d}_{2} )} ]}_{λ1}}{{[ ln \frac{I ( {d}_{1} )}{{I}_{0} ( {d}_{1} )} - ln \frac{I ( {d}_{2} )}{{I}_{0} ( {d}_{2} )} ]}_{λ3}} = \frac{{[ ln \frac{I ( {d}_{1} )}{I ( {d}_{2} )} ]}_{λ1}}{{[ ln \frac{I ( {d}_{1} )}{I ( {d}_{2} )} ]}_{λ3}} \qquad [ Mathematical Equation 41 ]$$

Accordingly, the second equation R2 may be expressed as a formula for the first) wavelength (λ₁) and the third wavelength (λ₃).

For example, it is assumed that the first wavelength (λ₁) value is 525 nm, the second wavelength (value is 660 nm, and the third wavelength (13 value is 950 nm.

Then, by substituting the molar extinction coefficient at each wavelength into the first equation R1 and the second equation R2, it may be expressed as Mathematical Equation 42 below.

$$[ Mathematical Equation 42 ]$$

$${R}_{1} = \frac{δ {A}_{660}}{δ {A}_{950}} = \frac{\begin{matrix}( 3.2269 \times {10}^{3} - SpO 2 \times 2907.4486 ) + \\ HbA 1 c \times \\ ( 7.8211 \times {10}^{4} + SpO 2 \times 2907.4486 )\end{matrix}}{\begin{matrix}( 602.083 + SpO 2 \times 601.917 ) + \\ HbA 1 c \times \\ ( 1.6527 \times {10}^{5} - SpO 2 \times 601.917 )\end{matrix}}$$

$${R}_{2} = \frac{δ {A}_{525}}{δ {A}_{950}} = \frac{\begin{matrix}( 3.5157 \times {10}^{4} - SpO 2 \times 4335 ) + HbA 1 c \times \\ ( 5.54 \times {10}^{5} + SpO 2 \times 4335 )\end{matrix}}{\begin{matrix}( 602.083 + SpO 2 \times 601.917 ) + HbA 1 c \times \\ ( 1.6527 \times {10}^{5} - SpO 2 \times 601.917 )\end{matrix}}$$

Next, the computation unit 130 may combine the first equation R1 and the second equation R2 to convert the concentrations of glycated hemoglobin (HbA1c) and arterial blood oxidation saturation (SpO2) into the formulas for the first equation R1 and the second equation R2, respectively, as shown in Mathematical Equation 43 below.

$$[ Mathematical Equation 43 ]$$

$$HbA 1 c = f ( {R}_{1} , {R}_{2} )$$

$$SpO 2 = f ( {R}_{1} , {R}_{2} )$$

By applying the first equation R1 and the second equation R2 to the function (f) in Mathematical Equation 43, the concentrations of glycated hemoglobin (HbA1c) and arterial blood oxidation saturation (SpO2) may be converted to Mathematical Equation 44 below.

$$[ Mathematical Equation 44 ]$$

$$HbA 1 c = \frac{- {aR}_{1} + {bR}_{2} - c}{{dR}_{1} - {eR}_{2} + f}$$

$$SpO 2 = \frac{{gR}_{1} - {hR}_{2} - i}{{jR}_{1} - {kR}_{2} + l}$$

In the mathematical equation above, a to l are positive real values.

When the concentrations of glycated hemoglobin (HbA1c) and arterial blood oxidation saturation (SpO2) are calculated using the first equation R1 and the second equation R2 to which the molar extinction coefficients at the first wavelength (λ₁) with a size of 525 nm, the second wavelength (λ₂) with a size of 660 nm, and the third wavelength (λ₃) with a size of 950 nm described above as an example are applied, it may be converted to Mathematical Equation 45 below.

$$[ Mathematical Equation 45 ]$$

$$HbA 1 c = \frac{- 3.96 {R}_{1} + 0.62 {R}_{2} - 14.71}{174.98 {R}_{1} - 87.93 {R}_{2} + 211.95}$$

$$SpO 2 = \frac{30.43 {R}_{1} - 2.7 {R}_{2} - 5.34}{5.97 {R}_{1} - 2.95 {R}_{2} + 7.56}$$

Accordingly, according to the third embodiment of the present disclosure, by acquiring the intensity of light measured by the light detection unit 120 installed on the body of the measurement subject and applying the ratios R1 and R2 of each acquired intensity value of light to the generated Mathematical Equation 44, the concentrations of glycated hemoglobin (HbA1c) and arterial blood oxidation saturation (SpO2) may be calculated. In addition, the calculated concentration of glycated hemoglobin (HbA1c) may be used to estimate blood sugar level.

In the third embodiment of the present disclosure, the ratio of each absorbance when transmitted through two LEDs having different wavelengths is generated as a mathematical equation, and three LEDs are applied to derive percentages for unknown, namely glycated hemoglobin (HbA1c) and arterial blood oxidation saturation (SpO2), using the ratio equation for the generated absorbance, but are not limited thereto. To increase the accuracy of detecting the concentration of glycated hemoglobin (HbA1c) and arterial blood oxidation saturation (SpO2) in a portion of the body, it is possible to install additional LEDs.

As such, the system 100 for non-invasive measurement of glycated hemoglobin according to the third embodiment of the present disclosure may non-invasively measure the concentration of glycated hemoglobin (HbA1C) using LED lights with three different wavelengths and the intensity change rate of light. The concentrations of glycated hemoglobin (HbA1C) and arterial blood oxidation saturation (SpO2) may be measured accurately and easily through the absorbance of LED light calculated using the Beer-Lambert Law.

Hereinbefore, although preferred embodiments of the present disclosure have been illustrated and described, it will be appreciated by those skilled in the pertinent technical field that various modifications and variations may be made without departing from the scope and spirit of the present disclosure as described in the claims below.

National Research Development Project supporting the Present Invention
Project Serial No.: 1711116583
Project No.: 2015R1A5A7037615
Department: Ministry of Future Creation and Science
Project management (Professional) Institute: National Research Foundation of Korea
Research Project Name: Leading research center support project Convergence field(CRC)
Research Task Name: Modular Smart Fashion Platform Research Center
Contribution Ratio: 1/2
Project Performing Institute: Kookmin University
Research Period: 2017 Mar. 1˜2022 Feb. 28
   National Research Development Project supporting the Present Invention
Project Serial No.: 1711107086
Project No.: 2019R1F1A1062317
Department: Ministry of Science and ICT
Project management (Professional) Institute: National Research Foundation of Korea
Research Project Name: Basic research support project
Research Task Name: Color space-based Visual-MIMO technology convergence research using color principles and learning algorithms
Contribution Ratio: 1/2
Project Performing Institute: Kookmin University
Research Period: 2019 Jun. 1˜2022 Feb. 28

[Description of Reference Numerals]

| | |
|---|---|
| 100: System for non-invasive | |
| measurement of glycated hemoglobin | |
| 110: Plurality of LED modules | 111: First LED module |
| 112: Second LED module | 113: Third LED module |
| 120: Light detection unit | 130: Computation unit |
| 140: Control unit | |

What is claimed is:

1. A method for non-invasive measurement of glycated hemoglobin, the method comprising:
irradiating a measurement subject with first to third lights having different wavelength values by means of first to third LED modules positioned on one side of the body of the measurement subject;
using a light detection unit positioned corresponding to the first to third LED modules to detect first to third derived lights derived from the first to third lights by passing through the measurement subject;
generating first and second ratio equations, respectively, for first and second derived light sets composed of two of the first to third derived lights; and
calculating concentrations of glycated hemoglobin (HbA1c) and arterial blood oxygen saturation (SpO2) of the measurement subject by combining the first and second ratio equations.

2. The method of claim 1, wherein the light detection unit is positioned at an opposite side or on the same side surface relative to positions of the first to third LED modules.

3. The method of claim 1, wherein one side of the body of the measurement subject comprises a site where capillaries existing under the skin are able to be sensed, depending on a thickness of the skin.

4. The method of claim 1, wherein the generation comprises generating the first and second ratio equations using Photon-Diffusion Theory or Beer-Lambert Law.

5. The method of claim 4, wherein the generation comprises generating an attribute equation for each wavelength of the first to third derived lights according to the Photon-Diffusion Theory, and generating the first and second ratio equations using the attribute equation for each derived light of the first and second derived light sets.

6. The method of claim 5, wherein the generation comprises generating an equation for transmittance or reflectance including a total absorption coefficient and a scattering coefficient for each wavelength of the first to third derived lights as the attribute equation.

7. The method of claim 6, wherein the generation comprises:
applying the total absorption coefficient and scattering coefficient to spherical geometry to express the transmittance or reflectance of each of the first to third derived lights using a mathematical equation;
generating a ratio for the transmittance or reflectance for each derived light of the first derived light set using the first ratio equation; and
generating a ratio for the transmittance or reflectance for each derived light of the second derived light set using the second ratio equation.

8. The method of claim 5, wherein the calculation comprises:
generating first and second conversion formulas by applying the glycated hemoglobin (HbA1c) and the arterial blood oxygen saturation (SpO2) as unknown to each of the first and second ratio equations;
applying coefficient values acquired corresponding to first to third wavelength ranges to each of the first and second conversion formulas; and
combining the first and second conversion formulas to convert the concentrations of the glycated hemoglobin (HbA1c) and the arterial blood oxygen saturation (SpO2) into each functional formula regarding the first and second ratio equations.

9. The method of claim 4, wherein the generation comprises applying attribute ratios regarding each derived light of each of the first and second derived light sets and the first to third derived lights measured by the light detection unit to the Beer-Lambert Law to generate the first and second ratio equations.

10. The method of claim 9, wherein the generation comprises:
generating the first ratio equation representing a ratio of absorbance for each derived light of the first derived light set by applying the Beer-Lambert Law; and
generating the second ratio equation representing a ratio of absorbance for each derived light of the second derived light set by applying the Beer-Lambert Law.

11. The method of claim 10, wherein the calculation comprises applying the first to third derived lights measured by the light detection unit to the first and second ratio equations to compute the concentrations of the glycated hemoglobin (HbA1c) and the arterial blood oxygen saturation (SpO2) of the measurement subject.

12. The method of claim 11, wherein the calculation comprises:
generating first and second conversion formulas by applying the glycated hemoglobin (HbA1c) and the arterial blood oxygen saturation (SpO2) as unknown to each of the first and second ratio equations;
applying a molar extinction coefficient when applying first to third wavelengths to each of the first and second conversion formulas; and
combining the first and second conversion formulas to convert the concentrations of the glycated hemoglobin (HbA1c) and the arterial blood oxygen saturation (SpO2) into each functional formula regarding the first and second ratio equations.

13. A system for non-invasive measurement of glycated hemoglobin, the system comprising:
first to third LED modules positioned on one side of the body of a measurement subject and respectively irradiating the measurement subject with first to third lights having different wavelength values;
a light detection unit positioned corresponding to the first to third LED modules to detect first to third derived lights derived from the first to third lights by passing through the measurement subject; and
a computation unit that generates first and second ratio equations, respectively, for first and second derived light sets composed of two of the first to third derived lights and calculates concentrations of glycated hemoglobin (HbA1c) and arterial blood oxygen saturation (SpO2) of the measurement subject by combining the first and second ratio equations.

14. The system of claim 13, wherein the light detection unit is positioned at an opposite side or on the same side surface relative to positions of the first to third LED modules.

15. The system of claim 13, wherein one side of the body of the measurement subject comprises a site where capillaries existing under the skin are able to be sensed, depending on a thickness of the skin.

16. The system of claim 14, wherein the computation unit generates the first and second ratio equations using Photon-Diffusion Theory or Beer-Lambert Law.

17. The system of claim 16, wherein, when using the Photon-Diffusion Theory, the computation unit calculates the concentration of the glycated hemoglobin using transmittance when the light detection unit is positioned at the opposite side, and calculates the concentration of the glycated hemoglobin using reflectance when the light detection unit is positioned on the same side surface.

18. The system of claim 16, wherein the computation unit calculates the concentration of the glycated hemoglobin using the Beer-Lambert Law and using absorbance when the light detection unit is positioned at the opposite side.
